(19) United States
(12) Patent Application Publication
(10) Pub. No.: US 2005/0032684 A1
(43) Pub. Date: Feb. 10, 2005

(54) Use of a peptide which activates guanylate-cyclase c for the treatment of respiratory airway problems via the airways, medicament, inhaltion devices and method of diagnosis

(76) Inventors: Yalcin Cetin, Hannover (DE); Yuksel Savas, Lengede (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/479,606

(22) PCT Filed: Jun. 5, 2002
(86) PCT No.: PCT/DE02/02040

(30) Foreign Application Priority Data
Jun. 5, 2001 (DE) 101-27-119.0

(51) Int. Cl. A61K 38/17

(52) U.S. Cl. 514/12

(57) ABSTRACT

The invention relates to the use of a guanylate cyclase C activated peptide for the treatment of respiratory airway problems and problems associated with ventilation disorder and/or mucous secretion disorders via the airways, in addition to a medicament which is fed via the airways. The invention also relates to an inhalation device which contains the medicament and a method for diagnosing the illnesses associated with inhalation disorders and mucous secretion disorders in the airways, by detecting a gualylate cyclase C activated peptide. The peptides which are used are guanylin, uroguanylin and lymphoguanylin or a heat resistant enterotoxin.

The invention relates to the use of a peptide which activates guanylate cyclase C for the treatment of respiratory tract disorders and disorders associated with impairments of ventilation and/or impairments of mucus secretion, to a relevant medicament, to an inhalation device and to a method for diagnosing the aforementioned disorders.

Obstructive impairments of ventilation are a serious clinical problem. They are associated with a constriction of the respiratory tract and thus an increase in the flow resistance, spasms of the bronchial muscles, edematous swellings of the bronchial wall and increased secretion (hypercrinia) of mucus of viscous consistency. The disorders associated with impairments of ventilation and/or impairments of mucus secretion include inter alia bronchial asthma, chronic bronchitis and mucoviscidosis.

No substances are currently available which have a lasting and efficient effect and which significantly improve the symptoms.

Secretolytics or mucolytics—which are also comprised among expectorants—in use are, inter alia, bromhexine, ambroxol, acetylcysteine and carbocisteine. The therapeutic value of these substances is, however, according to Mutschler, “Arzneimittel-wirkungen”, —Wissenschaftliche Verlagsgesellschaft mbH Stuttgart, 1996, doubtful.

The invention is based on the object of providing a novel effective means for the treatment of respiratory tract disorders and generally of disorders associated with impairments of ventilation and/or impairments of mucus secretion, it being intended that this means makes it possible in particular for bronchial mucus to be liquefied and transported away better.

The object is achieved by the use of a peptide which activates guanylate cyclase C for producing a medicament for the treatment of respiratory tract disorders and disorders associated with impairments of ventilation and/or impairments of mucus secretion via the airways, the medicament being formulated in such a way that the peptide is delivered on the air side of the respiratory tract, namely to the apical membrane of the mucosal epithelial cells.

It is also possible for a plurality of these peptides to be administered together or sequentially. It is equivalent to the use of these peptides themselves to use homologous peptides having substantially the same function, in particular those peptide variants having a sequence modification linked through deletion, insertion or exchange of one and/or more amino acids, sequence-extending attachment of one and/or more amino acids and/or chemical derivatization (especially of the terminal amino acids).

Pharmacologically acceptable derivatives are preferably amidated, acetylated, phosphorylated and glycosylated forms of the peptides and other post-translational derivatizations, including salts of these peptides and peptide derivatives.

It is possible to employ natural peptides or peptide mixtures which have been isolated for example from blood, lymph, urine or human or animal tissues and which ought to be purified, or synthetic or genetically manipulated (recombinant) peptides.

The peptide is, in particular, at least one of the peptides referred to as guanylin, uroguanylin and lymphoguanylin, or a heat-resistant enterotoxin. These peptides are known as such. It is also possible to use a peptide which is homologous with said peptides and has substantially the same function. Homologues mean in this connection peptides which substantially coincide with the sequences to be described below and are also included among the guanylin peptides by the skilled worker on the basis of their function and sequence homology. The skilled worker is aware that, for example, point mutations, deletions and insertions must not adversely affect the function of a peptide. Peptides modified in this way would therefore be included among the homologues.

Preferred at present is a guanylin peptide having 15 amino acids in the following sequence:
Seq. ID 1 (guanylin, 15 AA); PGTCEICAYAACTGC Pro-Gly-Thr-Cys-Glu-lle-Cys-Ala-Thr-Ala-Ala-Cys-Thr-Gly-Cys

A precursor molecule which has a length of 115 amino acids and which contains the above sequence is frequently likewise referred to as “guanylin”. Both peptides are suitable for the purposes of the invention, and the peptide with Seq. ID 1 is preferred and, as a relatively small peptide, can be delivered well by inhalation.

A 15 AA peptide having the sequence PGTCEICAYAACTGC was initially isolated from rat intestinal extracts and referred to as “guanylin”. After cloning and characterization of the cDNA for human guanylin it was evident that the guanylin is synthesized as precursor molecule having 115 AA (Seq. ID 4: MNAFLLFALCLLGAWAALAG GVTVQDGNFS FSLESVKKLK DLQEPQEPRV GKLRNFAPIPGEPVVPILCS NPNFPEELKP LCKEPNAQEI LQRLEEIAED PGTCEICAYAACTGC). It is now known that the bioactive protein circulating in the blood is not the precursor molecule but guanylin having 94 AA (proguanylin 22-115: VTVQDGNFS . . . PGTCEICAYA ACTGC). The term “guanylin” which is established in the literature describes both the 15 AA peptide and the longer 94 AA peptide.

Human uroguanylin is a peptide to which the following amino acid sequences has been assigned:
Seq. ID 2 (uroguanylin, 16 AA): NDDCELCVNVACTGCL Asn-Asp-Asp-Cys-Glu-Leu-Cys-Val-Asn-Val-Ala-Cys-Thr-Gly-Cys-Leu and was originally isolated from human urine, whence the name. U.S. Pat. No. 5,489,670 describes the isolation and synthesis of human uroguanylin and envisages use as laxative for constipation.

Uroguanylin was initially isolated as a 16 AA peptide (NDDC ELCVNVACTG CL) from urine. Cloning and characterization of the cDNA for human uroguanylin revealed a uroguanylin precursor molecule having 112 AA (Seq ID 5: MGCRAASGLL PGVAVVLLLL LQSTQSVYIQ YQGFRVQLES MKKLSDLEAQ WAPSPRLQAQSLLPAVCHHP ALPQDLQPVC ASQEASSI FKTLRTIA NDDC ELCVNVACTG CL). Elimination of the signal peptide produces an 86 AA uroguanylin (underlined sequence). The 16 AA peptide and the 86 AA peptide are referred to as uroguanylin.

Lymphoguanylin is a guanylin peptide which is expressed in lymphoid tissues and was found by Forte et al. (Forte et al. Endocrinology 1999, 140, 1800-1806). It comprises a peptide with a length of 15 amino acids and the following amino acid sequence:
Seq. ID 3 (lymphoguanylin, 15 AA): QEECELCINMACTGY Gln-Glu-Glu-Cys-Glu-Leu-Cys-lle-Asn-Met-Ala-Cys-Thr-Gly-Tyr

The lymphoguanylin precursor molecule comprises 109 amino acids (Seq. ID 6: MKVLALPMAV TAMLLIL AQN TQSVYIQYEG FQVNLDSVKK LDKLLEQLRG FHHQMGDQRD PSILC—DPALPSDLQPVCEN SQAVNIFRAL RYIN QEECELCINMACTGY).

The sequences reported for lymphoguanylin are derived from the opossum. The human sequence is not yet known. The 15 amino acid lymhoguanylin likewise activates human guanylate cyclase C.

It has been known for some time that the aforementioned peptides stimulate or activate guanylate cyclase, a G protein-coupled receptor which catalyzes the formation of cyclic guanosine monophosphate (cGMP) from guanosine triphosphate (GTP). Several guanylate cyclase-activating peptides were discovered in succession, and they are regarded as endogenous ligands of guanylate cyclase C. The first of these peptides was called guanylin (Currie, H. G. et al. Proc. Natl. Acad. Sci. USA 1992, 89, 947-951).

In the intestine, heat-stable enterotoxins—small peptides produced inter alia by pathogenic Escherichia coli strains—cause secretory diarrheas. These toxins also develop their effect through stimulation of the guanylate cyclase C which is expressed by intestinal epithelial cells. Like the heat-stable enterotoxins, the guanylin peptides lead to an increased electrolyte/water secretion on the intestinal mucosa. This means that guanylate cyclase C not only acts as receptor for heat-stable enterotoxins but also represents the genuine receptor for endogenous guanylin peptides.

A sequence suitable within the framework of the invention for a heat-stable enterotoxin is:
Seq. ID 7 (heat-stable enterotoxin): NSSNYCCELCCNPACTGCY (19 AA) from enteropathogenic E. coli.
   Mechanism of Action Common to Heat-stable Enterotoxins, Guanylin, Uroguanylin and Lymhoguanylin on the Intestinal Mucosa

In the intestinal mucosa, these guanylin peptides listed above and heat-stable enterotoxins lead, via activation of the joint receptor, to an increase in cGMP in the enterocytes. The increased cGMP level activates the cGMP-dependent protein kinase II (cGKII) in the enterocytes. This activated protein kinase phosphorylates, and thus opens, the CFTR chloride channel in the apical membrane of the enterocytes. This results in secretion of chloride ions and water into the lumen of the intestine. The CFTR chloride channel is now regarded as the final effector of the guanylin peptide signal transduction chain. These peptides therefore represent a direct regulator of the CFTR chloride channel.

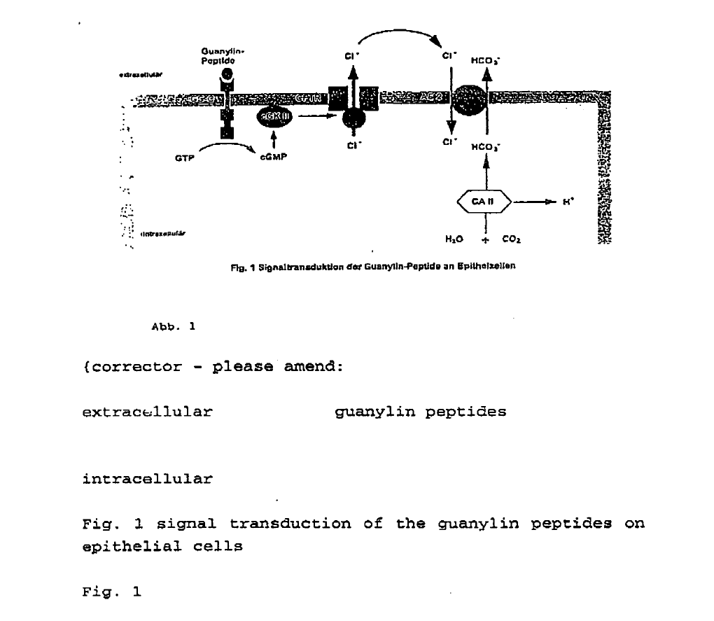

Pacticulsr attention is directed at the secretion of bicarbonate, which is also mediated by the guanylin peptides. According to findings to date, bicarbonate secretion takes place via a specific Cl⁻/HCO₃—exchanger (AE-2). It can be concluded on the basis of findings to date that the Cl⁻ secreted luminally via the CFTR is taken up again in the respective cells and exchanged for HCO₃—. It can thus be stated that the guanylin peptides play a central part in the regulation of Cl⁻ and HCO₃—in said enterocytes. The mechanism of action of the guanylin peptides is depicted in FIG. 1.

Said peptides circulate as endogenous activators in the blood. They can also be obtained from blood or hemofiltrate. Thus, DE 195 28 544 describes a guanylin peptide which was obtained from human blood and is intended for diagnostic, medical and commercial use as medicament. This peptide was referred to as GCAP-II. Because of the known effect of guanylin peptides on guanylate cyclase C (.see above), GCAP-II was intended specifically for the treatment of disorders associated with impairments of electrolyte transport in the cells. Use is to take place preferably by injection.

The endogenous activator guanylin is found at various sites in the body. Guanylin has been detected for example in the human pancreas (Kulaksiz et al, Histochem Cell Biol. (2001) 115, 131-145), in the kidney (Forte et al, Annu Rev. Physiol 2000, 62, 673-695), in the intestinal tract (Quian et alp Endocrinology 2000, 141, 3210-24) and in the lung (Cetin et al, Proc. Natl. Acad. Sci. USA, 92, 5925-5929, 1995).

The applicants have now found that the receptor common to heat-stable enterotoxins and guanylin peptides, the guanylate cyclase C, is localized in the mucosa of the airways and is expressed there to a large extent on the apical membrane (air side) on the respective epithelial cells, but not on the basolateral membrane (blood side). The receptor localized in the lung can therefore be stimulated exclusively via the airways, not via the bloodstream.

The mechanism of action at the cellular and molecular level is depicted in FIG. 1 which shows diagrammatically signal transduction of guanylin peptides on epithelial cells.

Guanylate cyclase (GC-C) is an enzyme-receptor complex which is localized as membrane protein exclusively in the apical cellular domain pointing toward the respiratory tract lumen. It is absent from the basolateral membrane of the cells (blood side) which is known to be in contact with the circulating blood.

Guanylin peptides which to the receptor (GC-C) via the lumen of the respiratory tract initiate a specific intracellular mechanism which contains various protein modules. The GC-C which is activated from outside by the guanylin peptides forms large amounts of cGMP from GTP inside cells. This second messenger (cGMP) activates a membrane-associated cGMP-dependent protein kinase of type II (cKGII) which undertakes the phosphorylation and thus activation of the CFTR protein on its regulatory (R) domain. CFTR is a membrane protein in the apical membrane of the epithelial cells and is an important chloride channel which, after activation, secretes chloride ions from the cell in the direction of the lumen of the respiratory tract. Owing to the ionic gradient produced in this way, water follows the secreted chloride ions and flows into the lumen of the respiratory tract. The water is derived from the epithelial cells and from the interstices between the cells (paracellular). Some of the chloride ions secreted into the lumen are taken up again in the cells; in an exchange, bicarbonate ions are secreted from the cells. This exchange of ions is brought about by the anion exchanger of type II (AE2). The AE2 protein is also localized in the apical membrane of the epithelial cells. Inside the cells, the bicarbonate ions are produced from water and carbon dioxide by the enzyme carbonic anhydrase of type II (CAII).

The membrane on the air side of the mucosal epithelial cells is thus the crucial site of signal reception, regulatory activity and electrolyte/water-secreting capacity in the respiratory tract.

The result of this mechanism of action of the guanylin peptides overall is that ions and fluid are secreted into the lumen of the respiratory tract, which crucially influence and determine the quality and flow properties of the bronchial mucus.

The following abbreviations are used in the figure: GC-C=guanylate cyclase C; cGKII=cGMP-dependent protein kinase of type II; CFTR=cystic fibrosis transmembrane conductance regulator; AE-2=anion exchanger of type II; CAII=carbonic anhydrase of type II.

Elucidation of the mechanism of action on which the invention is based was published in “Kulaksiz, H., Schmid, A., Hönscheid, M., Raraswamy, A., Cetin, Y., PNAS, May 2002, Vol. 99, pages 6796-6801”, “Kulaksiz et al., Histochem Cell Biol. (2001 115, 131-145”,

A central realization of the concept according to the invention is that activation of the receptor must take place through administration of the endogenous ligands specifically via the airways. The skilled worker must therefore adjust the delivery of the peptide or of the medicament containing the peptide in such a way that the peptide is delivered—as exclusively as possible—on the air side to the apical membrane of the respiratory tract and does not, for example, enter the bloodstream to a major extent. Targetea local therapeutic use in the respiratory tract is made possible precisely in this way, especially since the receptor is localized exclusively on the air side in the respiratory tract.

Delivery of peptides of the invention, namely the guanylate cyclase C ligands, via the airways comprises a directed and direct delivery to the receptor located on the air side. An increase in the concentration of the peptide in the blood through uptake through the lung, as is the intention on inhalation of other peptides (which have a systemic action, e.g. insulin), is to be strictly avoided in particular in this case.

Suitable means for this are available to the skilled worker. He is able to influence directed delivery to the air side via adjustment of the peptide concentration in the medicament formulation, the dosage and adjustment of the particle/droplet size within the formulation or the inhalation means so that virtually no peptide passes through to the blood side of the respiratory tract (to the basolateral membrane) and thus into the bloodstream. The optimal conditions can be found for each selected peptide in specific preliminary tests.

The invention makes possible a therapy with doses which are very much smaller than those which would be necessary to increase the concentration in the blood, while minimizing to eliminating the systemic side effects of the respective peptides.

Only on administration via the air do the heat-stable enterotoxins and said guanylin peptides lead to an adequate activation of the receptor guanylate cyclase C and thus to an increased secretion of fluid in the respiratory tract. On systemic administration in addition adverse reactions would be expected; for example, the enterotoxin leads to very unpleasant secretory diarrheas.

In addition, the peptides of the invention act as stimulants in the sense of secretolysis by breaking up the viscous mucus which is present in the airways, with the ionic composition and the pH of the fluid directly on the epithelial cells (“microclimatel”) being adjusted so that the viscous mucus increasingly “liquefies”.

The transport of mucus and microparticles out of the respiratory tract is made possible by epithelial cells which have on their apical side (air side) hairlike structures (cilia). The “cleaning” function is achieved by beats (towards the pharynx) of the cilia.

Since guanylin peptides also, besides their function of increasing electrolyte and water secretion, in particular activate the cilia-bearing epithelial cells, the frequency of ciliary beats on these cells is increased. The secretion and very small particles on the mucosa of the respiratory tract are thus transported away considerably more efficiently, in the sense of a concerted action, thus underlining the physiological and therapeutic importance of the guanylin peptides.

It should additionally be stated that said substances have a relaxing effect on smooth muscles in the wall of the bronchi and bronchioli. This leads overall to a considerable improvement in breathing.

The aforementioned newly found properties of the peptides of the invention act together synergistically for the purposes of the invention and lead to the very good effect of the peptides delivered through the airways for the treatment of the impairment and disorders mentioned at the outset.

The peptides of the invention can on the basics of these findings additionally be used for producing diagnostic aids for respiratory tract disorders and disorders associated with impairments of ventilation and/or impairments of mucus secretion.

In the first place, the peptides themselves are suitable for this purpose as reference substances for diagnosis. A lack/deficiency or an excess of these peptides for example in bronchial mucus, exudate or lavage may indicate the presence of impairments requiring treatment. Detection of the peptides is possible with conventional and known means such as spectroscopically, chromatographically or chemically.

A further possibility is to produce antibodies against the peptides of the invention for this detection by the skilled worker with the aid of methods and means customary for this purpose, which can then be employed in molecular biological or enzymatic assays.

A method for diagnosing said disorders therefore also contributes to achieving the object of the invention, wherein at least one of the peptides which activates guanylate cyclase C is detected, specifically and preferably in bronchial mucus, exudate, lavage, nasal secretion or saliva.

The detection can take place by detecting one of the sequences Seq ID 1 to ID 6
Seq. ID 1 (guanylin): PGTCEICAYA ACTGC
Seq. ID 4 (guanylin precursor molecule): MNAFLLFALCLLGAWAALAG GVTVQDGNFS FSLESVKKLK DLQEPQEPRV GKLRNFAPIP GEPVVPILCS NPNFPEELKPLCKEPNAQEI LQRLEEIAED PGTCEICAYA ACTGC
Seq. ID 2 (uroguanylin): NDDC ELCVNVACTGCL
Seq. ID 5 (uroguanylin precursor molecule): MGCRAASGLLPGVAVVLLLL LQSTQSVYIQ YQGFRVQLES MKKLSDLEAQ WAPSPRLQAQ SLLPAVCHHPALPQDLQPVC ASQEASSIFK TLRTIAN DDC ELCVNVACTG CL
Seq. ID 3 (lymphoguanylin): QEECELCINMACTGY
Seq. ID 6: (lymphoguanylin precursor molecule): MKVLALPMAVTAMLLIL-AQN TQSVYIQYEG FQVNLDSVKK LDKLLEQLRG FHHQMGDQRD PSILCSDPALPSDLQPVCEN SQAVNIFRAL RYIN QEECELCINMACTGY
Seq. ID 7 (heat-stable enterotoxin): NSSNYCCELCCNPACTGCY (19 AA) from enteropathogenic E. coli.

A test result is regarded as positive for the detection of an impairment if a concentration of at least one of the peptides which activate guanylate cyclase C is found to differ from comparison samples from healthy subjects.

The use according to the invention of the peptides further consists of formulating a medicament which is delivered via the airways and comprises at least one peptide which activates guanylate cyclase C. These peptides have been described in detail above.

Besides the peptide or the peptide mixture it is possible for at least one further active ingredient and, where appropriate, excipients and additives to be present in the medicament. Further active ingredients suitable in this connection are, for example, muscle-relaxing agents, local antibiotics, mainly for treating concurrently superimposed bacterial infections, or else additional mucolytics, secretolytics, antitussives or bronchiodilating substances. The selection will be made by the skilled worker on the basis of the particular needs for treating the disorders mentioned at the outset.

The medicament may be prepared in solid or liquid form and will be delivered via the airways by the user in a suitable way. It can for this purpose be administered using a commercially available atomizer or inhaler.

In a preferred embodiment, the medicament is in the form of an inhalable composition and comprises at least one propellant. Particularly suitable propellants are hydrochlorofluorocarbons. Suitable propellants are known to the skilled worker in this field. It is generally possible to use all suitable aerosol formers or else smoke formers. Depending on the excipient, an aerosol or a smoke is inhaled, with preference for an aerosol.

Finally, an inhalation device which comprises the medicament is provided to achieve the object, i.e. the medicament is present pre-packaged in the inhalation device. An inhalation device of this type may consist of a spray device, in particular a metering spray device or a metering inhaler (MDI, metered dose inhaler). Suitable inhalers are known to the skilled worker and described for example in U.S. Pat. No. 3,915,165, EP 166476 and U.S. Pat. No. 6,099,517. Ultrasonic nebulizers are also suitable.

The peptides of the invention should for administration firstly be converted into a finely dispersed form. For this purpose they can initially be converted into a solution or suspension and, where appropriate, stabilized in this form with pharmaceutically acceptable additions. It is possible to use acceptable surfactants, e.g. Tween®, for the stabilization. Also suitable, depending on the inhalation method, are commercially available emulsifiers approved as foodstuffs, e.g. lecithin. Further additives which may be present are salts, buffers, sugars, sorbitol, amino acids and many others. The complete preparation should be isotonic. To stabilize the fine dispersion it is likewise possible to provide a microencapsulation of the relevant peptides or an encapsulation in liposomes.

The peptides to be administered may also be in powdered form in the solid state, for example freeze dried, spray dried or crystallized from solution, and are then preferably mixed with dry hydrochlorofluorocarbons as propellant and aerosol former. On administration in the form of a powder it is possible to add solid additions, in particular stabilizers, for example sugars or sugar-like substances, lactose and the like.

Inhalation devices in which the aerosol formers or propellants on the one hand, and the actual medicament preparation on the other hand, are stored in different chambers and are delivered together in a preset dosage are also known. This avoids inaccurate dosage owing to inhomogeneity on storage.

The size of the particles to be inhaled is less critical than in many other applications because it is not intended that the peptides of the invention undergo transmembrane transport into the blood, but they must merely reach the receptor guanylate cyclase C which is localized apically in the lung. Particle sizes between 0.5 and 10 μm appear suitable.

The invention is illustrated by means of an example below:

The use of the peptides is to be illustrated by the example of “obstructive and restrictive impairments of ventilations”. These respiratory tract disorders are characterized by endobronchial obstruction with bronchospasm, mucosal edema and by hypersecretion of a viscous mucus (dyscrinia). These manifestations lead to the affected patient becoming thoroughly exhausted due to increased and incompetent effort of breathing. As restrictive component, gas exchange is considerably worsened by the mucosal edema, and oxygen uptake by the lungs is markedly reduced.

Use of the peptides aims at an effect contrary to these pathomechanisms. Inhalational administration leads to relaxation of the smooth muscles of the respiratory tract, so that the bronchial resistance and thus the patient's effort of breathing decreases. Exhaustion of the patient is lessened or prevented by making the effort of breathing easier.

Because of the electrolyte/water-secreting effects of these peptides, increased mobilization of water from the mucosa of the respiratory tract is induced and acts to reduce the mucosal edema (reduce swelling) and thus leads to improved breathing. The increased emergence of water from the mucosa reduces the dyscrinia, the viscous mucus liquefies and the transport away of the secretion is improved through increased beating of the cilia.

The peptides thus exert different functions which, in their combination and synergy, lead to a marked improvement in breathing.

1. The use of a peptide which activates guanylate cyclase C for producing a medicament for the treatment of respiratory tract disorders and disorders associated with impairments of ventilation and/or impairments of mucus secretion via the airways, the medicament being formulated in such a way that the peptide is delivered on the air side of the respiratory tract, namely to the apical membrane of the mucosal epithelial cells.

2. The use as claimed in claim 1, characterized in that the peptide is a natural or recombinant guanylin, uroguanylin, lymphoguanylin or heat-resistant enterotoxin, or is a peptide which is homologous thereto and has substantially the same function, in particular such a peptide variant having a sequence modification linked through deletion, insertion or exchange of one and/or more amino acids, sequence-extending attachment of one and/or more amino acids and/or chemical derivatization, in particular of the terminal amino acids).

3. The use as claimed in claim 1, characterized in that the peptide includes one of the sequences Seq. ID 1 to Seq. ID 7.

4. The use of a peptide as indicated in claim 1 for producing a diagnostic aid for respiratory tract disorders and disorders associated with impairments of ventilation and/or impairments of mucus secretion.

5. A medicament in a preparation which is delivered via the airways to the apical membrane, characterized in that it comprises at least one peptide which activates guanylate cyclase C.

6. The medicament as claimed in claim 5, characterized in that the peptide is guanylin, uroguanylin, lymphoguanylin or a heat-resistant enterotoxin, or a peptide which is homologous to the latter and has substantially the same function, in particular such a peptide variant having a sequence modification linked through deletion, insertion or exchange of one and/or more amino acids, sequence-extending attachment of one and/or more amino acids and/or chemical derivatization, in particular of the terminal amino acids, or a peptide mixture comprising at least one of these peptides.

7. The medicament as claimed in claim 5, characterized in that at least one of the peptides comprises one of the sequences Seq. ID 1 to Seq. ID 7.

8. The medicament as claimed in claim 5, characterized in that, besides the at least one peptide, the medicament comprises as active ingredient at least one further active ingredient and, where appropriate, excipients and additives.

9. The medicament as claimed in claim 5, characterized in that the medicament is in the form of an inhalation means and comprises at least one propellant, at least one aerosol former or at least one smoke former.

10. An inhalation device comprising the medicament as claimed in claim 5.

11. The inhalation device as claimed in claim 10, characterized in that it comprises a spray device, in particular a metering spray device or a metering inhaler.

12. A method for diagnosing disorders which are associated with impairments of ventilation and impairments of mucus secretion in the respiratory tract through detection of at least one peptide which activates guanylate cyclase C.

13. The method as claimed in claim 12, characterized in that the detection is directed at at least one of the sequences Seq. ID 1 to Seq. ID 7.

14. The method as claimed in claim 12, characterized in that the peptide is detected in exudate, bronchial mucus or lavage.

15. The method as claimed in claim 12, characterized in that a concentration of at least one of the peptides which activate guanylate cyclase C differing from comparison samples from healthy subjects is regarded as a positive test result for the detection of an impairment.


SEQUENCE LISTING (7 sequences)

SEQ ID NO: 1 - protein, 15 aa, rat
PGTCEICAYAACTGC

SEQ ID NO: 2 - protein, 16 aa, Homo sapiens
NDDCELCVNVACTGCL

SEQ ID NO: 3 - protein, 15 aa, oppossum (lymphoid tissue)
QEECELCINMACTGY

SEQ ID NO: 4 - protein, 115 aa, rat or homo sapiens (guanylin)
MNAFLLFALCLLGAWAALAGGVTVQDGNFSFSLESVKKLKDLQEPQEPRVGKLRNFAPIPGEPVVPILCSNPNFPEELKPLCKEPNAQEILQRLEEIAEDPGTCEICAYAACTGC

SEQ ID NO: 5 - protein, 112 aa, homo sapiens
MGCRAASGLLPGVAVVLLLLLQSTQSVYIQYQGFRVQLESMKKLSDLEAQWAPSPRLQAQSLLPAVCHHPALPQDLQPVCASQEASSIFKTLRTIANDDCELCVNVACTGCL

SEQ ID NO: 6 - protein, 109 aa, oppossum
MKVLALPMAVTAMLLILAQNTQSVYIQYEGFQVNLDSVKKLDKLLEQLRGFHHQMGDQRDPSILCSDPALPSDLQPVCENSQAVNIFRALRYINQEECELCINMACTGY

SEQ ID NO: 7 - protein, 19 aa, escherichia coli
NSSNYCCELCCNPACTGCY